(19) United States
(12) Patent Application Publication
(10) Pub. No.: US 2023/0352153 A1
(43) Pub. Date: Nov. 2, 2023

(54) METHOD FOR THE AUTOMATIC ALLOCATION AND DISPLAY OF RESPONSIBILITY IN TELEMEDICINE PROCEDURES AND SYSTEM

(71) Applicant: Siemens Healthcare GmbH, Erlangen (DE)

(72) Inventors: Christian Kaethner, Freiburg (DE); Marcus Pfister, Bubenreuth (DE)

(21) Appl. No.: 18/139,433

(22) Filed: Apr. 26, 2023

(30) Foreign Application Priority Data
Apr. 27, 2022 (DE) 10 2022 204 067.8

(51) Int. Cl. G16H 40/20; G16H 40/67

(52) U.S. Cl. CPC G16H 40/20; G16H 40/67

(57) ABSTRACT

For a particularly failsafe performance of telemedicine procedures a method is provided for the automatic allocation and display of responsibility in telemedicine procedures with at least one medical device, the procedures involving at least two members of medical staff and of which at least one first person being positioned in close proximity to the device (“locally”) and at least one second person being positioned remotely from the device, with the following steps: planning and/or retrieving an operational sequence of procedural steps for the performance of the telemedicine procedure, requesting or retrieving an information profile in each case for at least the first and second person, wherein the information profile for the performance of the procedure contains relevant information on the respective person, ascertaining a competency classification of the at least first and second person for each procedural step in the telemedicine procedure on the basis of the information profiles, and displaying an allocation of responsibility determined from the competency classifications ascertained for the at least first person and second person in relation to one another for at least one procedural step which is currently being carried out or is pending.

### CROSS REFERENCE TO RELATED APPLICATIONS

This application claims the benefit of DE 10 2022 204 067.8 filed on Apr. 27, 2022, which is hereby incorporated by reference in its entirety.

### FIELD

Embodiments relate to a method for the automatic allocation and display of responsibility in telemedicine procedures with at least one medical device and involving at least two members of medical staff.

### BACKGROUND

In modern medical technology, minimally invasive interventions involve the performance of treatments (for example placement of stents) or diagnoses (for example detection of stenoses) by way of objects introduced into hollow organs of the body (instruments, guidewires, stents, devices). Navigation into the individual vascular branches takes place by rotation and advancement of a guidewire or catheter, which is introduced at a puncture site in the patient's groin.

If such procedures are supported by a robotic system (for example by the Corindus CorPath GRX® vascular robot, see https://www.corindus.comkorpath-grx/how-it-works), the robot semi-automatically or automatically takes over the manipulation of the objects. An operator (for example a physician) remotely controls the movement by an operating unit. Depending on the application, the operator may be located in close proximity to the robot, for example in the same room (“locally”) or remote from the robot, for example in a corresponding room in a far-distant hospital (“remotely”). In the second case two persons, or even teams, situated at different locations must work together, one person or team locally with the patient and one person or team remotely for remote control of the robot. The responsibilities must be clearly distributed for this, for example by prior agreements and definition. In such a setting it may be that the persons or teams are not well established, for example do not know one another or do not even speak the same language. In the event of misunderstandings, errors or even conflicts between the persons or teams, the smooth running of the procedure is jeopardized and there may be serious consequences for the patient. If safety mechanisms for the synchronization of responsibilities between the persons or teams are incorporated, then in the event of time-critical steps this may bring about long delays and likewise result in the procedure or the patient being jeopardized.

### BRIEF SUMMARY AND DESCRIPTION

The scope of the present invention is defined solely by the appended claims and is not affected to any degree by the statements within this summary. The present embodiments may obviate one or more of the drawbacks or limitations in the related art.

Embodiments provide systems and methods that in the case of telemedicine procedures performed by persons or teams positioned at different locations permit a fast and error-free performance.

A method is provided for the automatic allocation and display of responsibility in telemedicine procedures with at least one medical device, the procedures involving at least two members of medical staff and of these at least one first person being positioned in close proximity to the device (“locally”) and at least one second person being positioned remotely from the device. The method includes: planning and/or retrieving an operational sequence of procedural steps for the performance of the telemedicine procedure, requesting or retrieving an information profile in each case for at least the first and second person, wherein the information profile contains information on the respective person relevant to the performance of the procedure, ascertaining a competency classification of the at least first and second person for each procedural step in the telemedicine procedure on the basis of the information profiles, for example taking into account a weighting of the information, and displaying an allocation of responsibility determined from the competency classifications ascertained for the at least first person and second person in relation to one another for at least one procedural step that is currently being carried out or is pending, wherein a reciprocal influence between information at least on the first person and information on the second person and/or a correlation between information at least on the first person and information on the second person is taken into account in the determination. More than two persons may be involved, for example two teams each containing two or more persons, but at least one first person is always positioned on site and at least one second person remotely. The information, information profiles, competency classifications, and allocations of responsibility may therefore also relate to teams or the corresponding terms may relate to more than two persons. The method may be performed in whole or in part during the telemedicine procedure, and also in part prior to the start of the telemedicine procedure.

In the method the information profiles, the ascertainment of the competency classification for the steps of the procedure, and the automatic determination of the responsibilities may be used to ensure conflict-free and simple coordination between persons or teams also positioned at different locations in a telemedicine procedure, in order to ensure that the performance of the procedure is fast and safe for the patient. By evaluating the information profiles the responsibilities may be assigned automatically and in an evidence-based manner, for example also taking into account correlations and synergy effects. Thanks to the display every person or team knows very precisely what they are responsible for. The method results overall in better treatment and care of patients and also in a higher patient throughput. The automatic performance of the method prevents the misunderstandings and delays that occur when coordination is performed manually.

In an embodiment, the first person is assigned a first display unit and the second person a second display unit and the ascertained allocation of responsibility is displayed on both the first and the second display unit. In this way an individual display is provided in each case for at least the first and second person, that is an advantage if the persons are at different locations. In this way it is also possible for each person, independently of one another, to be given a display specifically matched to them. For example, these displays may differ depending on the person assigned. Besides the persons' individual preferences as regards the choice of color, image sharpness, etc., the display may show each person precisely what their responsibilities are. The elements affecting the other person in each case may also be wholly omitted or displayed so that it is clear that they are not relevant to the person (for example scaled down, grayed out, etc.). In this way it is clearly and unambiguously apparent for each person what they are and are not responsible for and misunderstandings and errors may be prevented.

For example, the first person is assigned a first operating unit and the second person a second operating unit, similarly to the display units. In this way the persons may, independently of one another and in accordance with their responsibilities for steps in the medical procedure, operate operating functions of the system, of the medical device or other devices. Other manual actions may also be performed, for example by the person located on site, that cannot be controlled by the operating unit (inserting/changing catheters, measuring the patient's vital functions, emergency procedures, etc.).

In an embodiment, where the respective person holds secondary responsibility a block is activated during the telemedicine procedure on the corresponding operating functions, for example on the medical device or on other devices, and/or where the respective person holds primary responsibility a release of corresponding operating functions, for example of the medical device or of other devices. In this way it is already automatically ensured that no inadvertent or deliberate operating errors occur, since an automatic block is imposed if a person is not responsible for an operating function. The activation occurs during the corresponding steps of the telemedicine procedure. Therefore, for example during a telemedicine procedure including a radiographically monitored navigation of an object in a patient's body in which the second person is (remotely) responsible for the navigation, the operation of the robotic system may be blocked (locally) for the first person. Conversely, the monitoring of a patient monitor may be disabled for the second person concurrently or even throughout the entire procedure and released for the first person.

To provide the persons with a better overview, the corresponding block and/or the corresponding release are expediently displayed on the respective display units. This may for example be implemented using color coding (red for blocked).

In an embodiment, the information profile of the persons includes information on their place of residence and/or their training and/or their specialization and/or their language and/or their level of experience and/or the number of similar procedures performed in the past. Such information has a major influence on the performance of individual steps of the procedure and on the operation of device functions of the medical device or of other devices and is accordingly used to ascertain the competency classification. Thus, for example a person located remotely cannot affect a puncture into the patient for the insertion of the guidewire, since they are not on site. For many procedural steps it may also be important to know how often in the past a person has performed these steps or the corresponding device operations and how much experience they have. Different languages may impede communication between persons and thus easily result in misunderstandings. In principle it is relevant to the procedure to know what training or specialization the person has. Thus, for example a surgeon will be more suitable for a surgical procedural step than a specialist in internal medicine and is accordingly prioritized in the classification.

In an embodiment, updates to the respective information profile of the persons or of the procedural steps are used to determine and/or display competency classifications and/or allocations of responsibility updated in real time. In this way it is possible to react to changes in real time during the procedure or during certain procedural steps and thus to optimize the performance of the procedure.

In an embodiment, at least one pretrained machine learning algorithm is used to ascertain the competency classification and/or the allocation of responsibility.

Communications and interactions between the persons are advantageously monitored or regulated on the basis of the competency classifications and/or the information profiles.

Embodiments also provide a system for the performance of a corresponding method, configured for the performance of a telemedicine procedure, the procedure involving at least two members of medical staff and of these at least one first person being positioned in close proximity to the device (“locally”) and at least one second person being positioned remotely from the device, with at least two procedural steps, including at least one medical device, a control unit for activating the system, a retrieval unit for retrieving the persons' information profiles, a determination unit for determining a competency classification of the at least two persons for the respective procedural steps on the basis of the information profiles, for example taking into account a weighting of the properties, a calculation unit for creating an allocation of responsibility for each procedural step, wherein a reciprocal influence between properties and/or a correlation between properties is taken into account, at least two display units, wherein a first display unit is assigned to a first person and a second display unit to a second person, and at least two operating units, wherein a first operating unit is assigned to a first person and a second operating unit to a second person.

In an embodiment, where the respective person holds secondary responsibility the activation unit is provided to block operating functions of the medical device or of other devices, and/or where the respective person holds primary responsibility to release operating functions of the medical device or of other devices. The system may advantageously also have a planning unit for planning the telemedicine procedure.

### BRIEF DESCRIPTION OF THE FIGURES

FIG. 1 depicts an operational sequence of steps of the method according to an embodiment.

FIG. 2 depicts a further operational sequence of steps of the method according to an embodiment.

FIG. 3 depicts a further operational sequence of steps of the method according to an embodiment.

FIG. 4 depicts a view of a system for the performance of the method according to an embodiment.

FIG. 5 depicts a view of a first and a second operating unit during the method according to an embodiment.

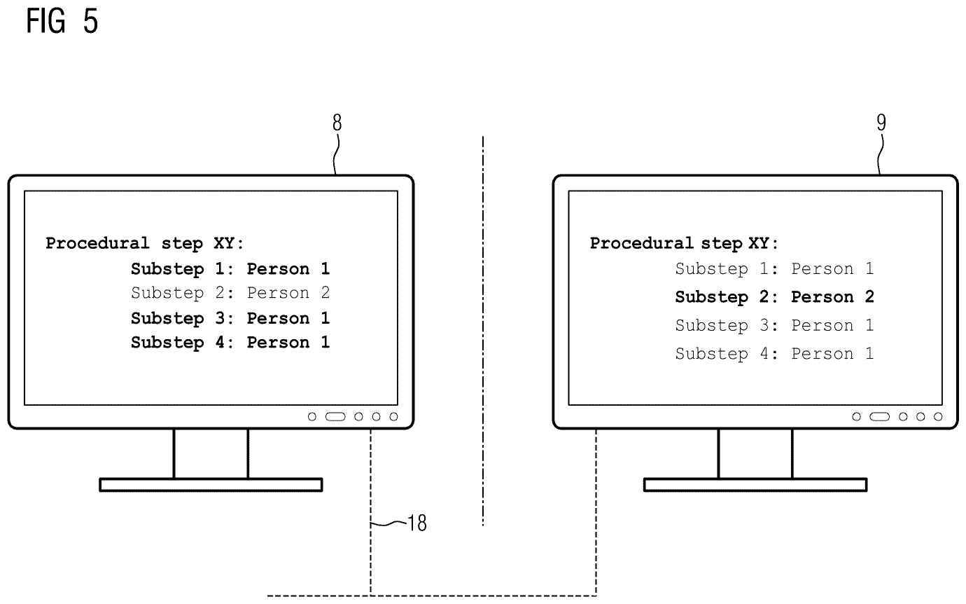

### DETAILED DESCRIPTION

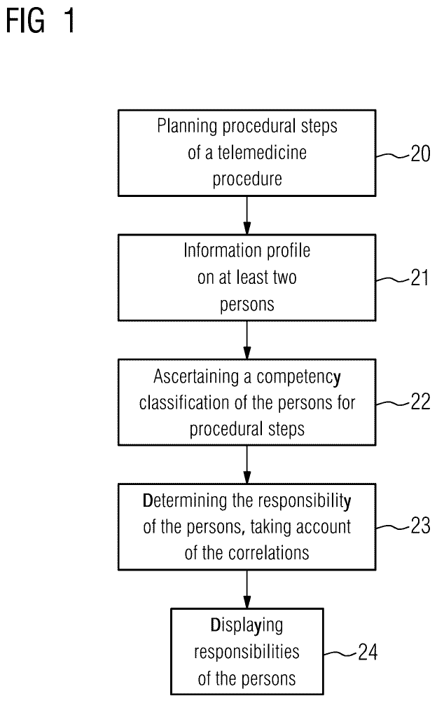

FIG. 1 depicts an operational sequence of steps of the method. The method optimizes cooperation in telemedicine procedures with at least one medical device, the procedures involving at least two members of medical staff and of these at least one first person being positioned in close proximity to the device (“locally”) and at least one second person being positioned remotely from the device. Thanks to the method, communication problems between the persons and other conflicts may be prevented, thereby ensuring a fast and orderly performance of the procedure that is safe for the patient. More than two persons may also be involved in the procedure and the persons may also for example, be split into two teams, of which a first team is positioned locally and a second team remotely. The information, information profiles, competency classifications and allocations of responsibility may in such a case also relate to the teams or the corresponding terms may relate to more than two persons or may be present for more than two persons.

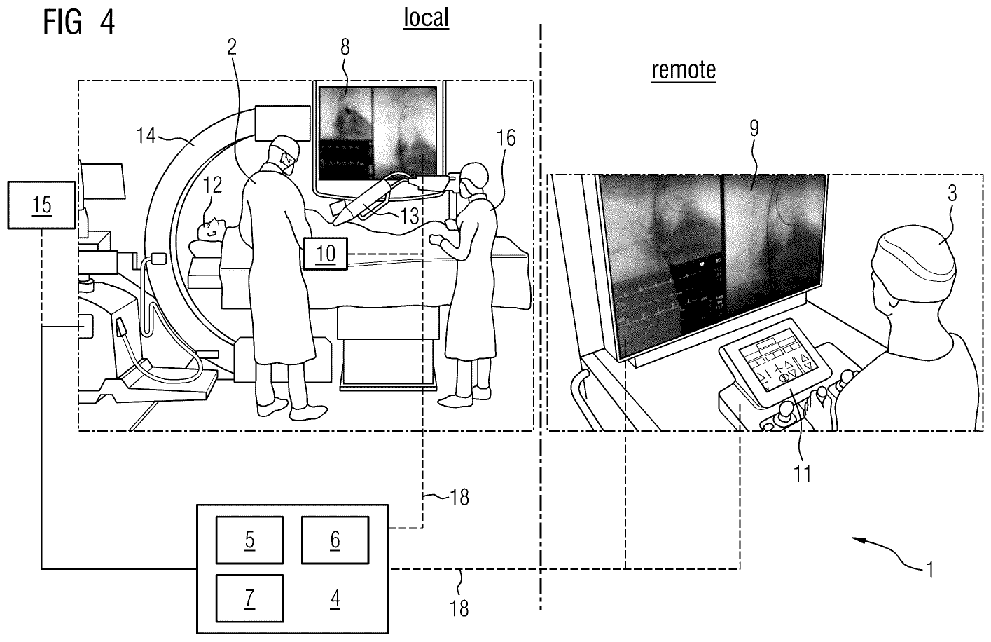

A standard telemedicine procedure may be performed by integrating remotely executed control of at least one of the devices involved. The telemedicine procedure may for example be formed by the remotely executed robot-assisted navigation of a medical object (for example guidewire, catheter, stent, device, instrument, etc.) through a hollow organ of a patient using radiographic monitoring (locally or remotely activated), for which a system with a robotic system for navigation and an X-ray device for radiographic monitoring may be used. Further devices, for example a plurality of devices, may also be involved. Any other telemedicine procedure is however also included in which members of the medical staff are positioned at different locations (locally and remotely). FIG. 4 depicts by way of example and system 1 for the performance of the method.

In a first step 20 an operational sequence of procedural steps for the performance of the telemedicine procedure is planned or an already preplanned procedure is retrieved from a memory unit or a database. The planning may be affected by a planning unit and on the basis of prior information, for example image datasets already present, patient data, etc. The telemedicine procedure in this case includes at least two consecutive procedural steps but may also include a plurality of procedural steps with more or fewer details. Besides the basic operational sequence of procedural steps, it is also possible for example to incorporate or plan the probability of emergency procedures, continuous patient monitoring, operational sequences extending over multiple steps or rare events. A procedural step may also be divided into two or more sub steps.

In a second step 21 an information profile for at least the first person and the second person is in each case requested or retrieved, wherein the information profile contains information or properties of the respective person relevant to the performance of the procedure. In one version an information profile of the corresponding person containing relevant information is already held in a memory unit or a database and merely has to be requested. Further information may however also be added, for example manually or automatically. Alternatively, or additionally, information in connection with the method may be requested, for example by asking the person to input the corresponding information into an input unit. Thus, for example a particular set of information may be relevant to each procedure, for example also depending on the type of procedure and the individual procedural steps. The totality of the information forms the respective information profile, for example in an orderly or even processed form.

The information may, for example, contain details of the person's medical training and/or the person's professional specialization, for example including in different degrees of detail. Thus, it is for example relevant to know whether the person is a physician or a technician or a nurse and whether the person specializes in particular medical disciplines or operation of devices. The information may also contain information on the level of experience (for example on clinical expertise as regards different devices or steps or patient monitoring) and/or the number of procedures performed by the person in the past. Information on the person's place of residence (for example locally or remotely) may also be included and may for example be updated at the start of the method. Information on the language of the person may also be included. Some of the information may be relevant in relation to the information about the respective other person(s) (positioned at another location) (for example language, level of training, level of experience, etc.). The information may also contain details about with which other person or persons the respective person has also previously collaborated, how often and in connection with which procedure. A retrieval unit 5 may for example be provided for retrieving information.

The first step 20 and the second step 21 may also be performed in the reverse order or even simultaneously.

In a third step 22, competency classifications are ascertained at least for the first person and for the second person for each procedural step in the telemedicine procedure on the basis of the information profiles. The competency classifications may for example be implemented as a type of aptitude assessment for the corresponding procedural steps, for example by evaluating the information profiles in respect of the procedural steps. This may be done in a variety of ways, for example by scoring, a (graduated) assessment or an assignment of points or marks in respect of the performance of the corresponding procedural step. The competency classification may be implemented in varying degrees of detail from very crude (for example 3 steps: “very suitable”, “fairly suitable” and “not suitable”) to very fine (for example more than 100 steps).

The information present in the information profiles in this case feeds into the competency classification. For example, information may also be weighted to a greater or lesser extent depending on the procedural step. For example, the information that a person is positioned remotely, for a procedural step to be executed on site (that requires direct contact with the patient, for example insertion of a needle, resuscitation procedure, etc.) may result in a “not suitable” competency classification. In such a case the weighting of this fact is very high, for example 100%. The competency classification may for example be performed by an ascertainment unit 6 of the system 1, for example using a machine learning (machine learning, deep learning, GAN, etc.) algorithm. Thanks to the use of machine learning algorithms the classification may be performed accurately, reliably, and fast. Machine learning algorithms may be trained using a plurality of examples.

In a fourth step 23 an allocation of responsibility of at least the first and second person for the procedural steps is determined, for example for the next currently envisaged procedural step or for multiple or for all planned procedural steps. As a result, it is precisely established for each person what they are responsible for as regards the respective procedural step and the details thereof, for example in the form of a distributed workflow. For example, the following may be provided in the case of a robot-aided navigation procedure with radiographic monitoring for the remotely positioned (second) person (or the team): where clinical expertise about the procedure is present, the second person is responsible for the general activation of the robotic system, general activation of the radiographic system, the selection of objects to be navigated and the instructions in respect of the objects to be inserted. Conversely, where sufficient expertise is present in respect of patient care/monitoring the (first) person located on site (or the team) is responsible for this and where a basic understanding is present about objects to be navigated (for example for the correct selection and for insertion/replacement under instruction) is responsible for on-site control and management of the robotic system and of the radiographic system.

When determining the allocation of responsibility, account may also be taken of a reciprocal influence between information on the first person and information on the second person and/or a correlation between information on the first person and information on the second person. If the remotely positioned second person for example has no prior knowledge of the radiographic system, the person positioned on site with sufficient experience in the field of controlling the radiographic system may take over; a remotely positioned second person with a great deal of experience would also be responsible for controlling the radiographic system. Different weightings of information may also be taken into account when determining responsibilities. Thus, for example different languages may be weighted less than a plurality of similar procedures performed jointly in the past.

The determination of the allocation of responsibility may for example be performed by a determination unit 7, for example likewise using a machine learning (machine learning, deep learning, GAN, etc.) algorithm. Thanks to the use of machine learning algorithms the classification may be performed particularly precisely, reliably, and fast. Machine learning algorithms may be trained using a plurality of examples.

In a fifth step 24 the allocation of responsibility for at least the first and second person is for example displayed on display units. The first person may here for example be assigned a first display unit 8 and the second person a second display unit 9. This is particularly advantageous, since the persons are positioned at different locations. Each person is now shown their assigned responsibility on their display unit, in particular the display therefore differs as a function of the person. The first person for example sees what they are themselves responsible for on the first display unit 8—see FIG. 4—and the second person sees what they are responsible for on the second display unit 9. The display may be performed for the procedural step that is currently being performed or that is to be performed in each case, for the following procedural step or else for all procedural steps. It may be provided that each person may exclusively see their own responsibility on their display. Alternatively, the respective person also sees the responsibility of the other person(s), but this for example differs visually from their own responsibility. The elements relating to the other person in each case may for example be displayed so that it is clear that they are not relevant to the person (for example scaled down, grayed out, etc.). In this way it is clearly and unambiguously apparent to each person what they are and are not responsible for and misunderstandings and errors may be prevented. In this way it is additionally also possible for each person to have implemented individual preferences on their display as regards choice of color, image sharpness, etc.

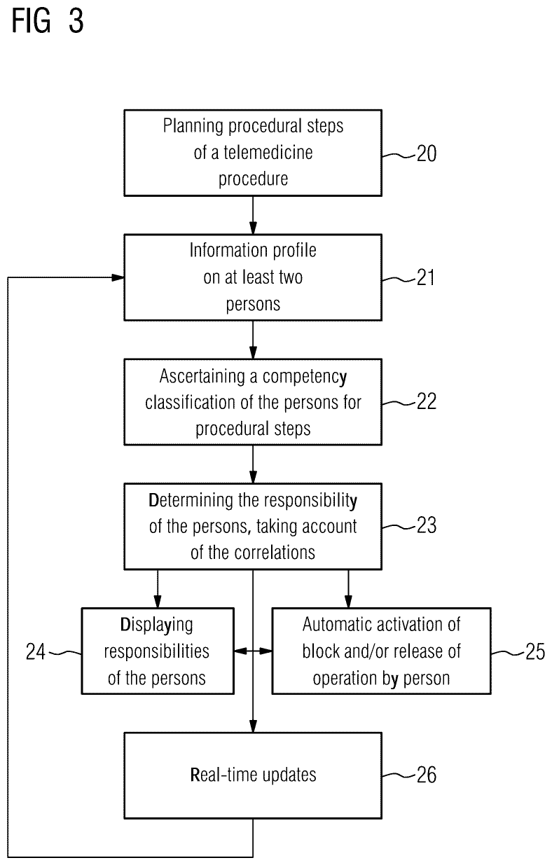

If changes (updates) to the information in respect of one or more persons occur or are initiated in the course of the procedure—see the seventh step 26 in FIG. 3—it may be provided that the respective information profile of the persons is updated and as a function thereof the competency classifications and/or the distribution of responsibility is determined and/or displayed in updated form. This may for example be performed live or in real time. Changes in the procedural steps (that for example emerge from the process or emergency cases or similar) may be used for updates. It may also be provided that the distributions of responsibility for the subsequent procedural step are not determined and/or displayed until during the operational sequence of a procedural step.

The persons may, analogously to the display units, also be assigned operating units, for example the first person is assigned a first operating unit 10 and the second person a second operating unit 11. The operating units may be designed for the operation and/or activation of operational functions of the device or devices involved and/or for inputs. In this way the persons may, independently of one another and in accordance with their responsibilities, activate operating functions of the system, of the medical device or other devices. The operating units 10,11 may for example be formed by touchpads, computer keyboards, icon bars, joysticks, operating buttons, switches, or a combination of these.

The method is activated and monitored automatically by a control unit 4 of the system. The control unit 4 has access to the various components of the system, for example to the determination unit 7 and the ascertainment unit 6 and to the first and second display unit 8, 9 and the first and second operating unit 10, 11. The control unit may have wireless or wired communication connections 18 and/or interfaces to all components.

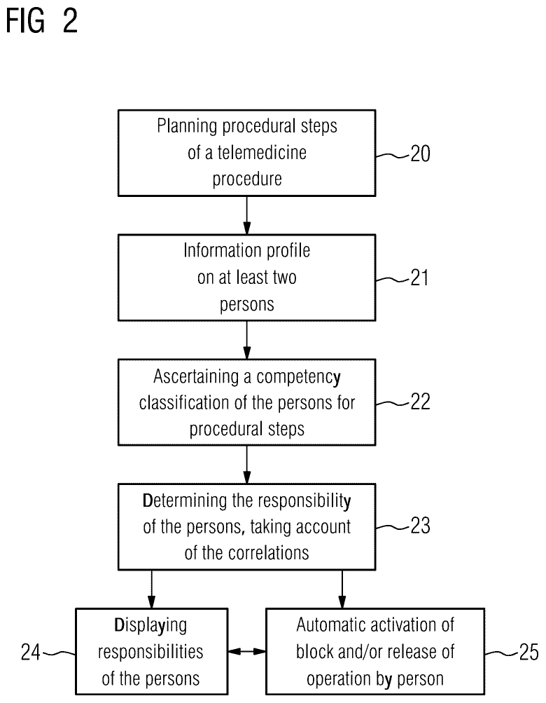

In a sixth step 25—shown in FIG. 2—an operating control for the persons may also be activated, for example via the operating units, during the procedure, besides the display of the distribution of responsibility. Thus, it may be provided that where the respective person holds secondary responsibility a block or lock on operating functions of the medical device or of other devices is activated, for example by disabling or blocking the corresponding functions on the operating units. This may likewise be activated and/or regulated automatically via the control unit 4.

Thus, for example in connection with a robotic intervention when an object (for example a catheter) is being changed manually by the first person 2, it may be provided that the second person 3 should not be able to perform any interaction with the robot and accordingly the corresponding operating function is for example blocked on the second operating unit 11 (or for example generally). If, for example in a critical situation for the patient, emergency procedures have to be performed by the first person 2 (for example resuscitation), the operation of any operating functions on the second operating unit may be completely blocked for the second person, until for example an input or release by the first person 2 has taken place. Operating functions for one person in each case may also be blocked or released by the respective other person, as long as this is provided for in principle. This may be the case for example if one of the persons has a significantly higher competency classification (for example because of a great deal of experience) than the other person. In addition, for example in the event that the persons have performed many procedures jointly in the past and trust one another, the respective responsible person may remove the block for the non-responsible blocked person and enable operation for them. On the other hand a responsible person may also block a non-responsible person by an input, for example if the responsible person has a very much higher competency classification.

Further aspects, for example how well communication between the persons works, may also be taken into account, for example whether functions are or are not disabled for a person. Thus, for example in the case of two persons (or teams) who are working together for the first time, when the second person has responsibility the same function may be blocked for the first person, whereas for persons familiar with one another, who for example have performed a plurality of similar procedures jointly in the past, both persons may access the functions, since it must be assumed that the non-responsible person will ask first before they actuate functions.

The system 1 for the performance of the method (FIG. 4) has, besides the control unit 4, medical devices in the form of a radiographic system 14 and a robotic system 13. The robotic system 13 is designed for the semi-automatic or fully automatic navigation of an object, for example guidewire, catheter, instrument, or stent, in a hollow organ of a patient 12, the radiographic system 14 for the radiographic monitoring of such navigation or generally for the acquisition of X-ray images of the patient. The system 1 is configured for the performance of a telemedicine procedure in which a first person 2 (or else a team consisting of two or more persons as shown) is positioned on site with the patient and a second person 3 (or else a team consisting of multiple persons) is remote from the patient, for example in another hospital, another city or even another country. The first person is assigned a first display unit 8 and a first operating unit 10 for operation and activation of operating functions of the robotic system 14 and of the radiographic system 13 and of other associated devices. The second person 3 is assigned a remotely positioned second display unit 9 and a remotely positioned second operating unit 11 for operation and activation of operating functions of the robotic system 14 and of the radiographic system 13 and of other devices. The control unit 4 for the activation of the method has wireless and/or wired communication connections 18 and interfaces to the components of the system. The control unit 4 has access to the various components of the system, for example to the determination unit 7 and the ascertainment unit 6. Further devices necessary for the performance of the medical procedure may be assigned, for example a patient monitor system for monitoring the patient's vital functions, an ECG, an ultrasound device, etc. More than one display unit may also be assigned per person and more than one operating unit (keyboard, smart device, foot switch, etc.).

The medical procedure is performed for the examination or treatment of the patient 12.

Various scenarios are also conceivable in respect of the use of the information or the information profile in connection with the method. For example, it may be provided that the control unit 4 exclusively accesses the distribution of responsibility in order to take corresponding display or interaction measures. It may also be provided that additional differentiations based on the competency classification are used in order to take dedicated measures. Alternatively, it is also possible to access the information or the information profile, for example as a weighting factor for the selection of the suitable measures.

Furthermore, account may also be taken in connection with the method of whether there are major differences between the persons or teams as regards the competency classification. Significantly different information may be displayed to the persons on the display units, wherein the management of the overall workflow is the responsibility of the control unit 4 and is adhered to thereby. The following examples are conceivable:

A very experienced (for example more than 100 procedures) second person (remote) with a great deal of knowledge about the devices being used and the procedure for example receives a suggestion from the control unit as to which level of detail is displayed to them. In addition, such a second person may even be given more freedom as regards settings or in an extreme example may even be awarded complete control (within certain basic conditions).

A particular (lower) degree of detail is for example suggested or even prescribed for a very inexperienced (for example fewer than 10 procedures) first person (local) with basic knowledge about the devices being used and the procedure, in order to ensure an appropriate information content, but also in order to influence the procedure as little as possible or to present it so that it corresponds to the level of knowledge. Possibilities for alternatives or even setting options would in this case be very strictly limited for the first person.

For example, the system 1 may further be designed to provide or if appropriate also block interaction mechanisms. For example, further suitable acoustic or visual displays may also be provided.

The system 1 is designed to capture general communication requirements, to evaluate them and to convert them into corresponding display and interaction measures.

Embodiments provide for a particularly failsafe performance of telemedicine procedures. A method is provided for the automatic determination and display of competencies in telemedicine procedures with at least one medical device, the procedures involving at least two members of medical staff and of which at least one first person being positioned in close proximity to the device (“locally”) and at least one second person being positioned remotely from the device, with the following steps: planning and/or retrieving an operational sequence of procedural steps for the performance of the telemedicine procedure, requesting or retrieving an information profile in each case for at least the first and second person, wherein the information profile contains information on the respective person relevant to the performance of the procedure, ascertaining a competency classification of the at least first and second person for each procedural step in the telemedicine procedure on the basis of the information profiles, in particular taking into account a weighting of the information, and displaying an allocation of responsibility determined from the competency classifications ascertained for the at least first person and second person in relation to one another for at least one procedural step that is currently being carried out or is pending, wherein in the determination a reciprocal influence between information at least on the first person and information on the second person and/or a correlation between information at least on the first person and information on the second person is taken into account.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present invention has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for automatic allocation and display of responsibility in a telemedicine procedure with at least one medical device, the telemedicine procedure involving at least two persons of medical staff of which at least one first person is positioned locally to the at least one medical device and at least one second person is positioned remotely from the at least one medical device, the method comprising:
planning an operational sequence of procedural steps for a performance of the telemedicine procedure;
retrieving an information profile for the at least one first person and the at least one second person, wherein a respective information profile contains information on a respective person relevant to the performance of the telemedicine procedure;
ascertaining a competency classification of the at least one first person and the at least one second person for each procedural step in the telemedicine procedure based on at least the information profiles; and
displaying an allocation of responsibility determined from the competency classifications ascertained for the at least one first person and at least one second person in relation to one another for at least one procedural step that is being carried out or is pending, wherein the determination takes into account a reciprocal influence between information for at least the first person and information for at least the second person, a correlation between information for at least the first person and information for at least the second person, or the reciprocal influence and the correlation.

2. The method of claim 1, wherein ascertaining includes a weighting of the information.

3. The method of claim 1, wherein the at least one first person is assigned a first display unit and the at least one second person is assigned to a second display unit, and a display of the determined allocation of responsibility is provided both on the first display unit and on the second display unit.

4. The method of claim 3, wherein a first display of the determined allocation of responsibility on the first display unit and a second display on the second display unit differ as a function of the assigned persons.

5. The method of claim 1, wherein when a person of the at least two persons holds secondary responsibility a block is activated on operating functions of the at least one medical device or of other devices or where the person of the at least two persons holds primary responsibility a release of operating functions of the at least one medical device or of other devices is activated.

6. The method of claim 5, wherein the block, the release, or the block and the release is displayed on respective display units for the at least two persons.

7. The method of claim 1, wherein the at least one first person is assigned a first control unit and the at least one second person is assigned a second control unit.

8. The method of claim 1, wherein the information profile of the at least two persons comprises at least one of information on:
their place of residence, their training, their specialization, their language, their level of experience, or a number of similar procedures previously performed.

9. The method of claim 1, wherein updates to the respective information profile of the persons or of the procedural steps are used in order to determine, display, or determine and display competency classifications or a distribution of responsibility updated in real time.

10. The method of claim 1, wherein the telemedicine procedure comprises at least one robot-aided navigation of an object in a hollow organ of a patient.

11. The method of claim 1, wherein at least one pretrained machine learning algorithm is used for the determination of the competency classification, the allocation of responsibility, or the determination of the competency classification and the allocation of responsibility.

12. The method of claim 1, wherein communications and interactions between the at least two persons are monitored or regulated on the basis of the competency classifications, the information profiles, or the competency classifications and the information profiles.

13. A system configured for a performance of a telemedicine procedure, the telemedicine procedure involving at least two persons of medical staff of which at least one first person is positioned locally to a medical device and at least one second person is positioned remotely from the medical device, the telemedicine procedure including at least two procedural steps, the system comprising:
the medical device;
a control unit configured to activate the system;
an ascertainment unit configured to ascertain a competency classification of the at least two persons for the respective procedural steps based on respective information profiles for the at least two persons that include respective information on a respective person relevant to a performance of the at least two procedural steps;
a calculation unit configured to determine an allocation of responsibility for each procedural step, wherein a reciprocal influence between the information, a correlation between the information, or a reciprocal influence and a correlation between the information is taken into account for the determination,
at least two display units, wherein a first display unit is assigned to the at least one first person and a second display unit to the at least one second person, and
at least two operating units, wherein a first operating unit is assigned to the at least one first person and a second operating unit to the at least one second person.

14. The system of claim 13, wherein where when a respective person holds secondary responsibility the control unit is configured to block operating functions of the medical device or of other devices and where the respective person holds primary responsibility, the control unit is configured to release the operating functions of the medical device or of other devices.

15. The system of claim 13, further comprising:
a planning unit for planning the telemedicine procedure and a retrieval unit for retrieving information profiles of the at least two persons.

16. The system of claim 13, further comprising:
at least one data transmission path for connecting the first operating unit and the second operating unit to the system.
